(19) United States
(12) Patent Application Publication
(10) Pub. No.: US 2024/0053345 A1
(43) Pub. Date: Feb. 15, 2024

(54) POLYPEPTIDES BINDING SELECTIVELY HEPARIN OR HEPARAN SULFATE GLYCOSAMINOGLYCANS AND CELL-PENETRATING POLYPEPTIDES COMPRISING THE SAME

(71) Applicants: SORBONNE UNIVERSITE, Paris (FR); CENTRE NATIONAL DE LA RECHERCHE SCIENTIFIQUE, Paris (FR); ECOLE NORMALE SUPERIEURE, Paris Cedex 05 (FR); INSTITUT NATIONAL DE LA SANTE ET DE LA RECHERCHE MEDICALE (INSERM), Paris (FR); College DE France, Paris Cedex 05 (FR)

(72) Inventors: Sandrine Sagan, Vitry sur Seine (FR); Alain Joliot, Paris (FR); Astrid Walrant, Paris (FR); Ludovic Carlier, Montrouge (FR); Sébastien Cardon, Paris (FR)

(73) Assignees: SORBONNE UNIVERSITE, Paris (FR); CENTRE NATIONAL DE LA RECHERCHE SCIENTIFIQUE, Paris (FR); ECOLE NORMALE SUPERIEURE, Paris Cedex 05 (FR); INSTITUT NATIONAL DE LA SANTE ET DE LA RECHERCHE MEDICALE (INSERM), Paris (FR); COLLEGE DE FRANCE, Paris Cedex 05 (FR)

(21) Appl. No.: 18/257,542

(22) PCT Filed: Dec. 17, 2020
(86) PCT No.: PCT/IB2020/001111

§ 371 Date: Jun. 14, 2023

(51) Int. Cl. G01N 33/574; C07K 14/47

(52) U.S. Cl. CPC G01N 33/57492; C07K 14/4725; G01N 2400/40; A61K 38/00

(57) ABSTRACT

The present invention relates to a polypeptide X1X2-RK-X3X4-KK-X5X6X7X8X9X10-KR-X11X12 SEQ ID NO: 1 able to bind selectively to cells expressing proteoglycans comprising glycosaminoglycans as heparin or heparan sulfate. The linkage of said polypeptide to an internalization peptide enables the polypeptide to be internalized.

The present invention further relates to a conjugated polypeptide comprising said polypeptide and a biomarker, to a cell penetrating polypeptide comprising said polypeptide, optionally a linker and a cell internalization peptide and a chimeric polypeptide comprising said cell penetrating polypeptide and one or more biological cargos.

The present invention relates to a polypeptide able to bind selectively to cells expressing proteoglycans comprising glycosaminoglycans as heparin or heparan sulfate. The linkage of said polypeptide to an internalization peptide enables the polypeptide to be internalized.

Homeoproteins (HPs) are autonomous transcription factors that are active during development and in adulthood. These proteins are found ubiquitously in plant and animal cells. HPs have paracrine activities and can travel from cell to cell through unconventional transfer or transduction pathway. Their intracellular activity implies a direct access of the traveling proteins to the cytosol and nucleus of recipient cells. HP atypical paracrine activity relies on common structural features shared by all HPs, and includes independent and distinct secretion and internalization regions. These regions reside in the 60-residue DNA-binding homeodomain (HD) that define the HP family. HDs are organized as three stable helices while the N- and C-terminal ends are variable and mostly unfolded. The third helix is responsible for the internalization activity of the protein while the secretion property requires a motif spanning both second and third helices. Notable is that the internalization region from the drosophila Antennapedia HP sequence is a cationic hexadecapeptide called Penetratin (RQIKIWFQNRRMKWKK). Penetratin is a cell-penetrating peptide (CPP). CPPs are a large family of peptides generally containing 5-30 amino acids and own the characteristic of spontaneously crossing the cell membrane (Derossi, D. & Prochiantz, A. Trojan peptides: the penetratin system for intracellular delivery. Trends Cell Biol. 8, 84-87 (1998)). Their positive charges intrinsically represent the major common properties of CPPs. The first CPPs Penetratin (Pen) and Tat are respectively from Antennapedia (Antp) transcription factor and the trans-activator protein of transcription of human immunodeficiency virus-1 (HIV-1).

CPPs interact with the different biomolecules at the cell surface. Cell-penetrating peptide internalization is strongly dependent on the presence of glycosaminoglycans (GAGs) both in vitro (Bechara et al. (2015) Massive glycosaminoglycan-dependent entry of Trp-containing cell-penetrating peptides induced by exogenous sphingomyelinase or cholesterol depletion. Cell Mol Life Sci 72(4):809-820.) and in vivo (Nakase I, Konishi Y, Ueda M, Saji H, Futaki S (2012) Accumulation of arginine-rich cell-penetrating peptides in tumors and the potential for anticancer drug delivery in vivo. J Control Release 159(2):181-188).

Cell membrane is decorated with diverse proteoglycans consisting of a core protein and a large number of linear polysaccharides, termed glycosaminoglycans (GAGs). GAGs are covalently linked to the core protein and negatively charged due to their sulfated groups and carboxyl groups.

GAGs are composed of different repeating disaccharide units. The disaccharide is composed of uronic acid derivatives (GlcA or IdoA) and an N-acylated or N-sulfated hexosamine.

GAGs are classified into the following four categories based on core disaccharides structures: heparan sulfate (HS), chondroitin sulfate (CS) and dermatan sulfate (DS), keratin sulfate (KS) and hyaluronic acid or hyaluronan (HA).

Heparin and heparan sulfate (HS) are composed of a repetition of glucuronic acid (GlcA) and iduronic acid (IdoA) with N-acetyl, N-sulfate and O-sulfate substitutions. Heparin is the structural analogue of HS but with higher sulfation.

Chondroitin sulfate (CS) is composed of a chain alternating N-acetylgalactosaminesulfate and glucuronic acid (GlcA). Sulfation rate vary within the CS subtype: CS-A and CS-C are mono-sulfated, CS-D and CS-E are di-sulfated. Dermatan sulfate (DS) is one subtype of chondroitin sulfate (CS): CS-B. The difference is the epimerization of the glucuronic acid into iduronic acid in DS.

Keratan sulfate (KS) is composed of a chain alternating N-acetylglucosamine and galactose. Keratan sulfate is the only GAG that does not contain uronic acid.

Hyaluronan (HA) is the only GAG existing as a free status without being linked to a core protein. HA is not sulfated and is composed of a chain alternating glucuronic acid (GlcA) and N-acetylglucosamine.

The content of HS on cells generally ranges from 50%-90% while CS is the second-largest component, the ratio of HS and CS is usually found as 4:1 in the surface of vascular endothelium.

The potential of CPPs for therapeutic molecules delivery into cells has been emphasized following the natural CPPs discovery. The lack of selectivity of the CPP-based delivery system between healthy cells and cancer cells causes side effects which is one of the important obstacles that hamper their biomedical application (Lindberg, S., Copolovici, D. M. & Langel, Ü. Therapeutic delivery opportunities, obstacles and applications for cell-penetrating peptides. Ther. Deliv. 2, 71 82 (2011)).

Regarding HPs, it was reported that the homoprotein Otx2 (orthodenticle homolog 2) internalization is restricted in vivo to specific neurons of the brain in mouse. Secreted Otx2 accumulates in parvalbumin neurons through interactions with surrounding perineural nets enriched in disulfated chondroitin sulfate (CS) GAGs, and regulates plasticity in the developing mouse visual cortex (WO2010081975). The region responsible for the addressing of Otx2 to its target cells is constituted by a peptide sequence of 15 amino acids.

Considering these drawbacks, the Inventors have searched for other HPs comprising a CPP, which could selectively target specific cells.

The Inventors have discovered a new region of Engrailed-2 (En2) that may selectively bind some cancer cells. This region has sequence similarities with nuclear localization signals (NLS) but, in contrast to Otx2, the newly discovered region is highly enriched in basic aminoacids and amazingly this NLS region of En2 is not the homologue of the Otx2 sequence.

En2 is a transcription factor that plays a role in the patterning of metazoan embryos. En2 regulates in particular the formation of boundaries during development of the brain in vertebrates. En2 does not accumulate in parvalbumin neurons and the region preceding the homeodomain strongly differs from that of Otx2, suggesting a different GAG selectivity.

The third helix of the homeodomain of En2 is a CPP, and its sequence is similar to Penetratin (Sgadó, P.; Genovesi, S.; Kalinovsky, A.; Zunino, G.; Macchi, F.; Allegra, M.; Murenu, E.; Provenzano, G.; Tripathi, P. P.; Casarosa, S.; Joyner, A. L.; Bozzi, Y. Loss of GABAergic Neurons in the Hippocampus and Cerebral Cortex of Engrailed-2 Null Mutant Mice: Implications for Autism Spectrum Disorders. Experimental Neurology 2013, 247, 496-505. https://doi.org/10.1016/j.expneurol.2013.01.021.).

Interestingly, the Inventors have discovered that the NLS region or cell recognition peptide of En2 links specifically to cells expressing heparin or heparan sulfate, especially some cancer cells.

They have further shown that this region associated with a third helix of a CPP is also able to address to specific cells any cargos linked to this chimeric polypeptide.

The present invention relates to an isolated polypeptide defined by the sequence: RK-XXX-KK-XXXXXX-KR, in which X is an amino acid, each X being independent from each other, and wherein X is selected from the group arginine or histidine or lysine or asparagine or glutamine or tryptophan or serine or cysteine or threonine or methionine or proline, or aspartate or glutamate.

According to the preferred embodiment the sequence RK-XXX-KK-XXXXXX-KR, in which X is an amino acid, each X being independent from each other, and wherein X is selected from the group arginine or histidine or lysine or asparagine or glutamine or tryptophan or serine or cysteine or threonine or methionine or proline or aspartate or glutamate, and wherein at least three X are proline.

According to a preferred embodiment, the isolated polypeptide of the invention is: RKP-XX-KK-X-P-XXXX-KR P.

This isolated polypeptide according to the invention is a cell recognition polypeptide binding selectively heparin or heparan sulfate glycosaminoglycans.

The present invention further relates to the isolated polypeptide defined by the sequence:

(SEQ ID No 1)
X₁X₂-RK-X₃X₄-KK-X₅X₆X₇X₈X₉X₁₀-KR-X₁₁X₁₂,

in which:
X₁ represents none amino acid or arginine (R) or histidine (H) or lysine (K) or asparagine (N) or glutamine (Q) or tryptophan (W); if X₂ is nothing, X₁ is nothing;
X₂ represents none amino acid or serine (S) or cysteine (C) or threonine (T) or methionine (M) or asparagine (N) or glutamine (Q) or tryptophan (W);
X₃ represents a proline (P) or any amino acid;
X₄ represents lysine (K) or histidine (H) or arginine (R) or tryptophan (W) or asparagine (N) or glutamine (Q);
X₅ represents asparagine (N) or aspartate (D) or glutamate (E) or glutamine (Q);
X₆ represents a proline (P) or any amino acids;
X₇ represents asparagine (N) or aspartate (D) or glutamate (E) or glutamine (Q);
X₈ represents lysine (K) or histidine (H) or arginine (R) or asparagine (N) or glutamine (Q) or tryptophan (W);
X₉ represents glutamate (E) or aspartate (D) or asparagine (N) or glutamine (Q);
X₁₀ represents aspartate (D) or asparagine (N) or glutamate (E) or glutamine (Q);
X₁₁ represents a proline (P) or any amino acids;
X₁₂ represents arginine (R) or lysine (K), or histidine (H) or asparagine (N) or glutamine (Q) or tryptophan (W).

“None amino acid” means that the polypeptide of SEQ ID No. 1 does not comprise any amino acid on the defined position; for example, if X₁ represents none amino acid, then the SEQ ID No. 1 does not comprise any amino acid at the 1^{st} position and begins by the X₂ amino acid.

In a preferred embodiment,
X₁ represents arginine (R) or histidine (H) or lysine (K);
X₂ represents serine (S) or cysteine (C) or threonine (T) or methionine (M);
X₃ represents a proline (P);
X₄ represents lysine (K) or histidine (H) or arginine (R);
X₅ represents asparagine (N) or aspartate (D) or glutamate (E) or glutamine (Q);
X₆ represents a proline (P);
X₇ represents asparagine (N) or aspartate (D) or glutamate (E) or glutamine (Q);
X₈ represents lysine (K) or histidine (H) or arginine (R);
X₉ represents glutamate (E) or aspartate (D) or asparagine (N) or glutamine (Q);
X₁₀ represents aspartate (D) or asparagine (N) or glutamate (E) or glutamine (Q);
X₁₁ represents a proline (P);
X₁₂ represents arginine (R) or lysine (K) or histidine (H).

Preferably, the present invention relates to an isolated polypeptide defined by the sequence:

(SEQ ID No 1)
X₁X₂-RK-X₃X₄-KK-X₅X₆X₇X₈X₉X₁₀-KR-X₁₁X₁₂,

in which
X₁ represents none amino acid or arginine (R) or histidine (H) or lysine (K) or asparagine (N) or glutamine (Q) or tryptophan (W); if X₂ is nothing, X₁ is nothing;
X₂ represents nothing or serine (S) or cysteine (C) or threonine (T) or methionine (M) or asparagine (N) or glutamine (Q) or tryptophan (W);
X₄ represents lysine (K), or histidine (H) or arginine (R) or tryptophan (W) or asparagine (N) or glutamine (Q);
X₅ represents asparagine (N), or aspartate (D) or glutamate (E) or glutamine (Q);
X₇ represents asparagine (N) or aspartate (D) or glutamate (E) or glutamine (Q);
X₈ represents lysine (K) or histidine (H) or arginine (R) or asparagine (N) or glutamine (Q) or tryptophan (W);
X₉ represents glutamate (E) or aspartate (D) or asparagine (N) or glutamine (Q);
X₁₀ represents aspartate (D) or asparagine (N) or glutamate (E) or glutamine (Q);
X₁₂ represents arginine (R) or lysine (K) or histidine (H) or asparagine (N) or glutamine (Q) or tryptophan (W).

In a more preferred embodiment,
X₁ represents arginine (R) or histidine (H) or Lysine (K);
X₂ represents serine (S) or cysteine (C) or threonine (T) or methionine (M);
X₄ represents lysine (K), or histidine (H) or arginine (R);
X₅ represents asparagine (N), or aspartate (D) or glutamate (E) or glutamine (Q);
X₇ represents asparagine (N) or aspartate (D) or glutamate (E) or glutamine (Q);
X₈ represents lysine (K) or histidine (H) or arginine (R);
X₉ represents glutamate (E) or aspartate (D) or asparagine (N) or glutamine (Q);
X₁₀ represents aspartate (D) or asparagine (N) or glutamate (E) or glutamine (Q);
X₁₂ represents arginine (R) or lysine (K), or histidine (H).

According to one preferred embodiment of the invention, the cell recognition polypeptide is characterized by the sequence RSRKPKKKNPNKEDKRPR (SEQ ID No. 2). The SEQ ID No. 2 is the sequence of the NLS of Ent.

The present invention also relates to a conjugated polypeptide comprising the polypeptide of SEQ ID No. 1 or 2 and a biomarker conjugated at its N-terminal end or its C-terminal end or on a lateral position linked to an amino acid.

In the context of the present invention, the term “biomarker” refers to a detectable and optionally measurable substance in a biological entity such as a patient or isolated organs, tissues or cells.

A “biomarker” denotes any molecule or molecular complex used for labelling purpose, for example, fluorescent biomarker (fluorescein, rhodamine, Cy3, Cy5, nitrobenzoxadiazole (NBD)), radiolabeled biomarker (18F, 68Ga), biotin, organic (micelle, liposome, dendrimer, polymer), inorganic (gold, iron oxide, lanthanide ions, carbon, silica) or composite (core-shell, MOF-metal organic framework) nanoparticles.

More specifically, the conjugated polypeptide of the invention is a diagnostic biomarker both in vivo and in vitro to detect cells expressing heparin or heparan sulfate, especially cancerous cells.

The present invention thus further relates to:
said conjugated peptide for its use in the in vivo detection method of a cancer involving cells expressing heparin or heparan sulfate glycosaminoglycans;
use of said conjugated peptide, for the in vitro detection of cells expressing heparin or heparan sulfate glycosaminoglycans;
a composition to diagnose cancer involving cells expressing heparin or heparan sulfate glycosaminoglycans, characterised in that it contains said conjugated peptide.

Another object of the invention is an in vitro detection method of cells expressing heparin or heparan sulfate glycosaminoglycans in a biological sample from a patient comprising the steps of:
(i) put in contact biological sample and said conjugated peptide;
(ii) in vitro detection of the linkage between the conjugated peptide with the cells from the biological sample, the detection of this linkage denoting the presence of cells expressing heparin or heparan sulfate glycosaminoglycans in the biological sample.

The detection is measured in vitro.

The detection method is used to detect and to diagnose cancers and the presence of cells expressing heparin or heparan sulfate glycosaminoglycans in the biological sample indicates that the patient has a cancer or is a diagnostic of a cancer.

The general term “biological sample” for the purposes of the present description is a sample of cells from a patient.

According to another object, the present invention relates to a cell penetrating polypeptide, defined by the general formula (I) or (II):

X₁X₂-RK-X₃X₄-KK-X₅X₆X₇X₈X₉X₁₀-KR-X₁₁X₁₂-L-CIP (I)

CIP-L-X₁X₂-RK-X₃X₄-KK-X₅X₆X₇X₈X₉X₁₀-KR-X₁₁X₁₂ (II)

wherein X₁ to X₁₂ and their preferred embodiments are the same as those previously described; L is nothing or a spacer and CIP is a cell internalization peptide facilitating the cell penetration of the polypeptide.

The spacer can be a flexible linker with at least three atoms or a conformationally restricted linker with at least three atoms or an aliphatic flexible linker with five atoms or an hydrophilic flexible linker with nine atoms.

In a preferred embodiment, the spacer can be a glycine or a proline or an aminopentanoic acid or polyethylene glycol.

Several cell internalization peptide sequences can be used:

SQIKIWFQNKRAKIKK (SEQ ID No. 3), RQIKIWFQNRRMKWKK (Penetratin, SEQ ID No. 4), RRWWRRWRR (SEQ ID No. 5), RRWRRWWRR (SEQ ID No. 6), RRWWRRWWR (SEQ ID No. 7), RRRRRRRR (SEQ ID No. 8) and YGRKKRRQRRR (SEQ ID No. 9).

CIP sequences which have been described in the prior art (EP1795539, EP2579899) which are incorporated by reference.

According to one specific embodiment of this object, a biomarker, as defined before, can be added to the cell penetrating polypeptide of general formula (I) or (II) in N-terminal end, C-terminal end or on a lateral position linked to an amino acid.

Thanks to the cell recognition peptide, the cell penetrating polypeptide (CPP) can link to cells expressing heparin and heparin sulfate and can enter into cells with its cell internalization sequence (CIP).

Thus, another object of the invention relates to a chimeric polypeptide composed of the cell penetrating polypeptide of general formula (I) or (II) and a biological cargo. The cargo is coupled to the cell-penetrating peptide through an amide, maleimide, disulfide or triazole bond either at the N-terminal or C-terminal end or on an amino acid side chain.

The general term “biological cargo” denotes any molecule or molecular complex, that is desired to target to a target cell. The biological cargos that can be transported by cell penetrating polypeptides in accordance with the invention can be a nucleic acid or a polypeptide. In case of a peptide, a preferred embodiment for the biological cargo is the sequence KRAKLAK (SEQ ID No. 10).

Another object of the present invention is related to a pharmaceutical composition comprising a chimeric polypeptide according to the invention and an acceptable pharmaceutically vehicle.

Another object of the invention relates to the chimeric polypeptide of the invention and said pharmaceutical composition for use as a medicament.

In a specific embodiment, said chimeric polypeptide and said pharmaceutical composition are for use to treat cancer involving cells expressing heparin or heparan sulfate (HS) glycosaminoglycans.

The prior art has shown important roles of HS in oncogenic signaling. The deregulations of both HS and heparin sulfate proteoglycan HSPG are involved in solid tumors as well as hematological malignancies among prostate, breast, lung, leukemia, colorectal, pancreatic, ovarian, neuroblastoma, myeloma, carcinoma, gliobastoma, head/neck, melanoma, testicular germ cell, Wilm's tumor, yolk sac tumor, hepablastoma, bladder, sarcoma, endometrial, renal cell, non-Hodgkin's lymphoma. HS and HSPG are considered as good targets to treat cancers (Nagarajan et al., Heparan sulfate and heparin sulfate proteoglycans in cancer initiation and progression, Frontiers in endocrinology, 24 Aug. 2018).

Advantageously, the cell penetrating polypeptide of the invention combines the cellular internalization with the third helix and the GAG selectivity to target cells expressing HS.

The present invention will be better understood with the aid of the additional description which follows, which refers to non-limiting Figures and Examples illustrating the identification of a targeting polypeptide in accordance with the invention and the demonstration of its specificity of addressing.

### FIGURES

FIG. 1: Positions (a) and sequence alignments (b) of potential GAGs-binding motifs and internalization sequence in homeoprotein Engrailed 2 (En2) and Otx2. The 16 amino acids internalization sequence (conserved third helix) in HD of En2. The grey rectangle marks the heparin sulfate (HS)-binding motif in En2 and the chondroitin sulfate (CS)-binding motif in Otx2. The grey rectangle above Engrailed 2 proposes an alternative HS-binding motif En2 aligned to the one of Otx2.

FIG. 2: Quantification of the internalization of peptides, studied in GAGs-deficient cells CHO-745 (white bar), wild type cells CHO-K1 (light grey bar) and two ovarian adenocarcinoma cells CaOV-3 (dark grey bar) and SKOV-3 (black bar) after 1 h incubation at 37° C. Experiments were done in duplicate or triplicate and repeated at least three times independently. Significance was tested using a student's t-test (NS:p>0.05, *0.05>p>0.01, **0.01>p>0.001, ***0.001>p).

FIG. 3: Quantification of internalization of chimeric peptides P1, P3 and P5 in cells CHO-745 (a,b) and in CaOV-3 cells (c,d). Peptides were incubated with cells for 1 h along with heparin (a) or chondroitin sulfate-E (b). P1 was also incubated with CaOV-3 for 1 h along with heparin (c) and P3 incubated with CaOV-3 in the presence of CS-E (d) at different concentrations. Experiments were done in duplicate or triplicate and repeated at least three times independently.

FIG. 4: Quantification of P3 after SKOV-3 pre-treatment with sodium chlorate for 24 h or GAG-specific enzymes for 2 h (a) and P1 after SKOV-3 pre-treatment with GAG-specific enzymes for 2 h (b). Experiments were done in duplicate or triplicate and repeated at least three times independently. Significance was tested using a student's t-test (NS:p>0.05, *0.05>p>0.01, **0.01>p>0.001, ***0.001>p).

FIG. 5: The effect of heparin favoring P1 internalization can be reversed by addition of heparinase in CHO-K1.

FIG. 6: Cytotoxicity of peptides alone incubation at different concentrations in CHO-K1 (a) and hemolysis of P1 and P3 at 10 μM and 50 μM incubation in blood cells. (b) (***p<0.001).

### EXAMPLES

### Example 1: Role of Third Helix and GAGs for the Cellular Internalization

### Materials and Methods

### Tested Polypeptides

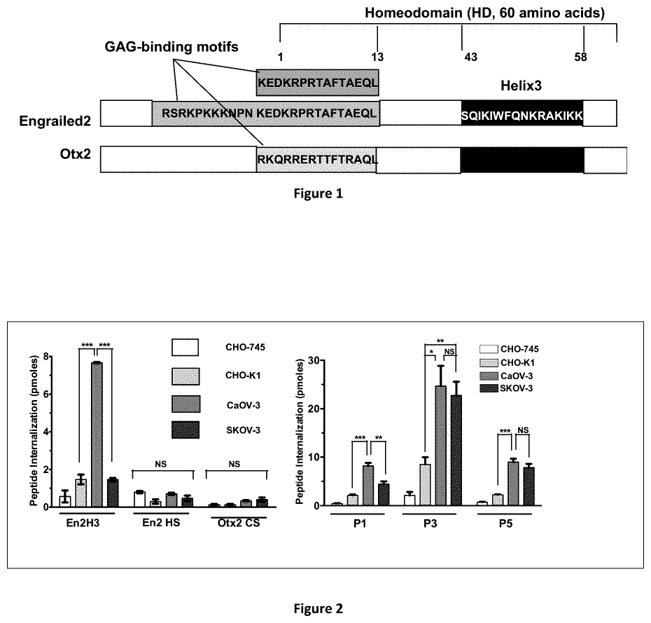

The chimeric polypeptide P1 have been obtained by synthetizing the third helix 43-58 residues of En2 HD (En2H3) as cell-penetrating part and by picking up a region from the upstream of En2 HD as a putative HS-binding motif (EnHS) to conjugate with En2H3 as represented on FIG. 1.

The inventors conjugated CS-binding peptide (OtxCS) with En2H3 to give potentially a CS-binding CPP (P3). Finally, they also synthesized P5 that is the peptide motif in En2 aligned to the CS-binding peptide from Otx2 conjugated to the En2H3.

TABLE 1
where G5 is Gly-Gly-Gly-Gly-Gly and Ac is an acetyl moiety

| | |
|---|---|
| P1 | Biotin-G₅-RSRKPKKKNPNKEDKRPR SQIKIWFQNKRAKIKK-NH₂ |
| P3 | Biotin-G₅-RKQRRERTTFTRAQL SQIKIWFQNKRAKIKK-NH₂ |
| P5 | Biotin-GGGG-KEDKRPRTAFTAEQL SQIKIWFQNKRAKIKK-NH₂ |
| En2H3 | Ac-SQIKIWFQNKRAKIKK-NH₂ |

### Cell Lines and Culture

Peptides listed in Table 1 were incubated separately in the following four ovarian cell types which have different GAGs types and expression levels: wild type CHO-K1 (expressing heparan sulfate (HS), chondroitin sulfate A (CSA) and C (CSC)), CHO-pgs A475 (CHO-745) (genetically modified to express only 5-10% HS and CS), two human ovarian adenocarcinoma cells CaOV-3 (overexpressing HS and CSE) and SKOV-3 (overexpressing CSE). Peptides were incubated with one million cells at 37° C. for 1 h.

Wild type Chinese Hamster Ovary (CHO-K1) cells and GAGs-deficient mutant CHO-745 cells which lack the xylosyltransferase needed for glycosaminoglycan (GAG) synthesis were grown in Dulbecco's modified Eagle's medium F-12 (DMEMF-12) with L-glutamine and 15 mM HEPES. HEK cells and HeLa cells were grown in DMEM with 4.5 g/L D-glucose and pyruvate. Two types of human ovarian cancer cell lines CaOV-3 and SKOV-3 were cultured in DMEM with Glutamax and McCoy's 5A medium, respectively. All complete culture medium was supplemented with 10% fetal bovine serum, penicillin (100,000 IU/L), streptomycin (100 mg/L). Cells were grown in a humidified atmosphere at 37° C. and 5% CO2.

### Results

The internalization results are shown in FIG. 2, En2H3 and three chimeric CPPs (P1, P3 and P5) entered significantly more in CHO-K1 or the two ovarian cancer cells than in CHO-745 cells. The quantities of all peptides increased as the order: CHO-745<CHO-K1<CaOV-3/SKOV-3. It indicates that GAGs play a favorable role in the cellular internalization of these peptides.

Comparing the internalization efficacies of the two GAG-binding peptides and the other four CPPs, it is observed that EnHS and OtxCS could not enter efficiently in any of the cell lines (FIG. 2) which illustrates that even though EnHS or OtxCS bind to HI and CSE efficiently, this interaction step is not sufficient for cell internalization. Besides, once acquiring the absolute quantities of internalized peptides taken from 1 million cells and taking account of the CHO cell volume (1 pL/cell), the inventors can also obtain the molar concentration of cellular peptides. For example, 1 pmol internalized peptides in 1 million cells mean 1 μM peptide inside the cell. FIG. 2 shows the internalization efficacy for each CPP ordered as P3>>P1≥P5≥En2H3 all four cell lines: P3 entered at 24 μM intracellular concentration, while the other three CPPs internalized at 7 μM in CaOV-3. P3 and the penetrating part En2H3 have similar Kd for HI (ITC result), while the internalization of P3 is much better than En2H3 alone. Therefore, the difference might derive from the specificity of OtxCS with disulfated CS. Since HS has been investigated in many studies and shown to be associated with the endocytosis pathway, this result somehow implies that CS-mediated pathway can also occur. More interestingly, all CPPs En2H3, P1, P3, P5 increased their internalization efficacy in CaOV-3 compared to CHO-K1, however, only P3 and P5 also apparently increased their cellular uptake in SKOV-3 (FIG. 2). The different behaviors of peptides P1, P3 and P5 in these two ovarian cancer cells might arise from their different levels of CSE and turn out the selective participation of CSE in P3 and P5 internalization. P5 exhibits lower cell uptake in both cancer cells compared to P3, which might result from a lower binding affinity of P5 for cell surface polysaccharides.

### Example 2—Role of GAGs in Specific Internalization Pathways

### Materials and Methods

### Tested Polypeptides

P1, P3 and P5 are used in this example within the sequences described in the table 1.

### Cell Lines and Culture

Tested Peptides were incubated separately in the following two ovarian cell types: CHO-745 and CaOV-3. The inventors incubated (7.5 μM) peptides with cells lines for 1 h along with exogenous heparin (HI) or chondroitin-4,6-sulfate (CSE) and quantified the internalized peptides by MS.

Cells culture are similar to the example 1.

### Results

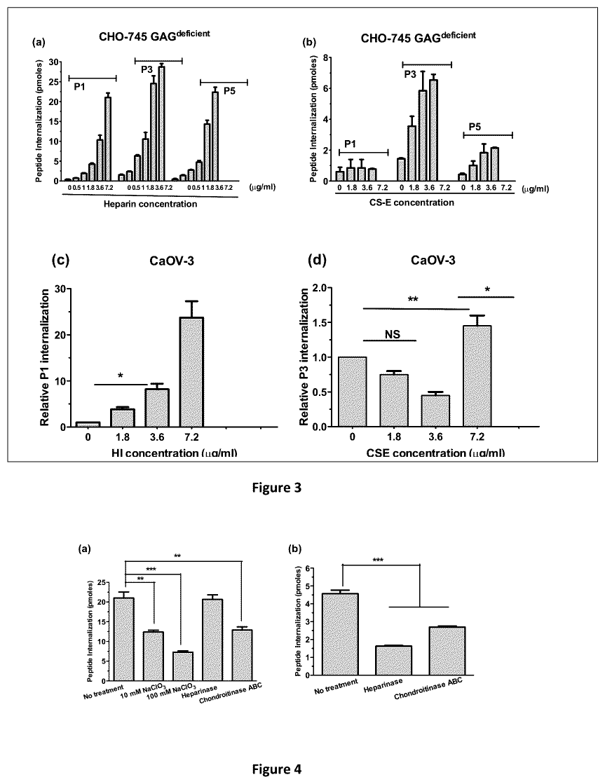

Desulfation or degradation of cell-surface HS and CS lead to a significant decrease in the internalization of P3 in SKOV-3. In the FIG. 3, the inventors further investigated the role of GAGs in specific internalization pathways by the adding exogenous GAGs to GAGs-deficient cells. Herein, they used P1, P3 and P5. As FIG. 3a shows, the three chimeric peptides increased internalization in CHO-745 by addition of exogenous HI according to concentrations. After co-incubation of 7.2 μg/ml HI with individual peptide in CHO-745, the cellular quantities of P1, P3 and P5 were around 21 pmol, 28 pmol and 22 pmol respectively which were even over the corresponding internalized quantities in CaOV-3 overexpressing HS. However, with the co-incubation of CSE in the same range of previous HI concentrations, FIG. 3b shows that CSE exogenous addition only resulted in an increase of P3 internalization to around 6.5 pmol and P5 internalization to 2.2 pmol at 7.2 μg/ml CSE concentration. By contrast, CSE addition made no effect on P1 internalization in CHO-745. Although P1 binds to CSE with a high binding affinity, it is not well internalized with CSE addition, which confirms again that binding to GAG is not sufficient to enter into cells. Exogenous HI promotes all three chimeric peptides internalization in GAGs-deficient cells while CSE specifically and efficiently enhances P3 cellular uptake only. The inventors further co-incubated HI or CSE with peptides in CaOV-3 cells whose GAGs (HS and CS) are overexpressed.

FIG. 3c shows the favorable effect of HI on the relative internalization of P1 in CaOV-3 cells, while CSE addition inhibited P3 internalization at the concentrations below 3.6 μg/ml but evokes P3 internalization at a concentration of 7.2 μg/ml (FIG. 3d). The results imply that exogenous CSE competed with endogenous cell-surface CSE to combine P3 at low concentrations of CSE, while over the threshold of CSE concentration, promotion of P3 cellular uptake occurred. These results imply that HI and CSE might play distinct roles in internalization, HI being more effective than CSE to evoke peptide cellular internalization probably with endocytosis. In summary, the sulfated groups and intact GAGs are partners for peptide internalization. Desulfation and fragments of GAGs lead to an irreversible impairment of peptides internalization efficacies. Exogenous addition of HI and CSE might result in a distinct effect on the GAGs-binding CPPs. HI could efficiently promote peptide uptake in GAGs-deficient cells or CaOV-3 cells at low concentrations while CSE only selectively enhances peptide internalization at high concentration and the plateau of effect is not as high as the one induced by HI.

### Example 3—GAG Sulfation is Required for GAG-Binding Peptide Internalization

### Materials and Methods

### Tested Polypeptides

P1 and P3 are used in this example within the sequences described in the table 1.

### Cell Lines and Culture

Tested Peptides were incubated separately in the following ovarian cell type: SKOV-3. A pre-treatment with sodium chlorate for 24 h or GAG-specific enzymes for 2 h.

Cells culture are similar to the example 1.

### Results

The inventors used different concentrations of chlorate to treat SKOV-3 and analyzed the internalization of P3. The result in FIG. 4 suggests that P3 internalization efficacy was dramatically reduced by preventing sulfation of cell surface GAGs in SKOV-3, the internalization reduction being sodium chlorate concentration-dependent. The inventors also checked that there is no cytotoxicity generated by 100 mM concentration of NaClO3. This result implies that GAG sulfation is required for GAG-binding peptide internalization. Not merely impairment of sulfation leads to significant P3 internalization decline, enzymatic GAGs degradation might also impact on peptide internalization. They used two specific enzymes: one is the blend of heparinases I, II and III that are extracted from Flavobacterium heparinum and hydrolyze the glycosidic bonds of glucosamine and uronic acids; the other one is chondroitinase ABC (ChABC) lyase purified from Proteus Vulgaris that specifically catalyzes the degradation of chondroitin 4-sulfate, chondroitin 6-sulfate containing (1-4)-β-D-hexosaminyl and (1-3)-β-D-glucuronosyl or (1-3)-α-L-iduronosyl linkages to disaccharides containing 4-deoxy-β-D-gluc-4-enuronosyl groups. FIG. 4a shows that CS degradation resulted in significantly curtailed internalization of P3 while heparinases cocktail treatment made almost no difference in P3 cellular uptake in SKOV-3.

Of outmost interest, FIG. 4b suggests that both heparinases cocktail and ChABC treatment in SKOV-3 induced a decrease in P1 internalization, however, a bigger effect was obtained with heparinases compared to ChABC treatment.

### Example 4—P1 Internalization Linked to Sulfate Heparane GAGs

### Materials and Methods

### Tested Polypeptides

P1 is used in this example within the sequence described in the table 1.

### Cell Lines and Culture

P1 was incubated in the CHO-745 ovarian cell type with different concentrations of heparinase. Cells culture are similar to the example 1.

### Results

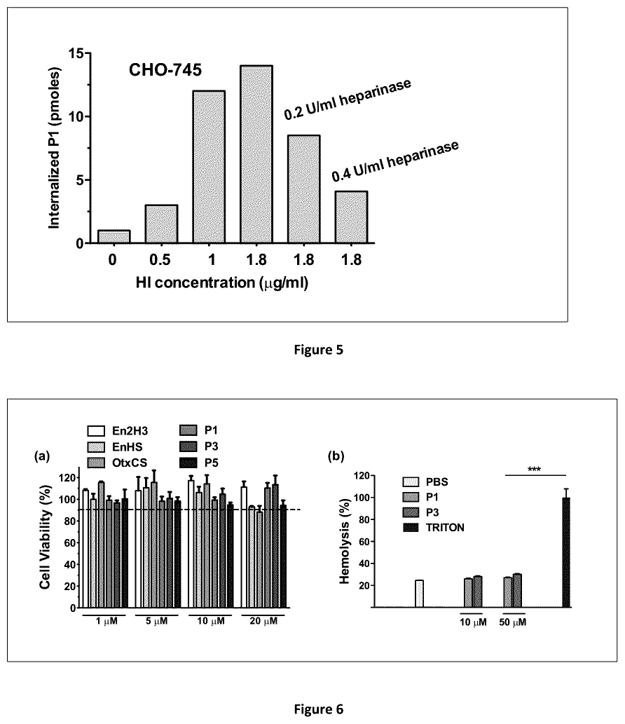

FIG. 5 shows that intact HI promoted P1 internalization in CHO-745 while degradation fragments of HI lost the ability, revealing that a polymer size of HI is required. In summary, the sulfated groups and intact GAGs are partners for peptide internalization. Desulfation and fragments of GAGs lead to an irreversible impairment of peptides internalization efficacies. Exogenous addition of HI and CSE might result in a distinct effect on the GAGs-binding CPPs. HI could efficiently promote peptide uptake in GAGs-deficient cells or CaOV-3 cells at low concentrations while CSE only selectively enhances peptide internalization at high concentration and the plateau of effect is not as high as the one induced by HI.

### Example 5— P1 and P3 are not Cytotoxic

### Materials and Methods

### Tested Polypeptides

P1 and P3 are used in this example within the sequences described in the table 1.

### Cell Lines and Culture

P1 and P3 were incubated separately in the CHO-K1 ovarian cell type with different concentrations of heparinase.

Cells culture are similar to the example 1.

### Results

The inventors tested cytotoxicity and hemolysis activity of the peptides incubated with CHO-K1 cells at different concentrations. In FIG. 6a, the percentage of cell viability after 1 h incubation of peptide alone suggests minimal cytotoxicity (less than 10%) caused by peptide even at 20 μM concentration. Moreover, incubation of the two chimeric peptides P1 and P3, as PBS, made no hemolysis of blood cells even at 50 μM for 2 h (FIG. 6b).

1. Polypeptide characterised in that it is defined by the sequence:
(SEQ ID No 1)
X₁X₂-RK-X₃X₄-KK-X₅X₆X₇X₈X₉X₁₀-KR-X₁₁X₁₂,
in which:
X₁ represents none amino acid or arginine (R) or histidine (H) or lysine (K) or asparagine (N) or glutamine (Q) or tryptophan (W); if X₂ is nothing, X₁ is nothing;
X₂ represents nothing or serine (S) or cysteine (C) or threonine (T) or methionine (M) or asparagine (N) or glutamine (Q) or tryptophan (W);
X₃ represents a proline (P) or any amino acids;
X₄ represents lysine (K), or histidine (H) or arginine (R) or tryptophan (W) or asparagine (N) or glutamine (Q);
X₅ represents asparagine (N), or aspartate (D) or glutamate (E) or glutamine (Q);
X₆ represents a proline (P) or any amino acids;
X₇ represents asparagine (N) or aspartate (D) or glutamate (E) or glutamine (Q);
X₈ represents lysine (K) or histidine (H) or arginine (R) or asparagine (N) or glutamine (Q) or tryptophan (W);
X₉ represents glutamate (E) or aspartate (D) or asparagine (N) or glutamine (Q);
X₁₀ represents aspartate (D) or asparagine (N) or glutamate (E) or glutamine (Q);
X₁₁ represents a proline (P) or any amino acids;
X₁₂ represents arginine (R) or lysine (K), or histidine (H) or asparagine (N) or glutamine (Q) or tryptophan (W).

2. Polypeptide as claimed in claim 1, wherein said polypeptide is a cell recognition polypeptide binding selectively cells expressing heparin or heparan sulfate glycosaminoglycans.

3. Polypeptide as claimed in claim 2, characterised in that it is defined by the sequence: RSRKPKKKNPNKEDKRPR (SEQ ID NO: 2).

4. Conjugated polypeptide comprising the polypeptide of claim 1 conjugated to a biomarker at its N-terminal end or its C-terminal end or on a lateral position linked to an amino acid.

5. Composition to diagnostic cancer involving cells expressing heparin or heparan sulfate glycosaminoglycans, characterised in that it contains a conjugated polypeptide according to claim 4.

6. Conjugated polypeptide according to claim 4 for its use in the in vivo detection method of a cancer involving cells expressing heparin or heparan sulfate glycosaminoglycans.

7. Use of a conjugated polypeptide according to claim 4, for the in vitro detection of cells expressing heparin or heparan sulfate glycosaminoglycans.

8. In vitro detection method of cells expressing heparin or heparan sulfate glycosaminoglycans in a biological sample from a patient comprising the steps of:
(i) put in contact biological sample and the conjugated polypeptide according to claim 4;
(ii) in vitro detection of the linkage between said conjugated polypeptide with the cells from the biological sample, the detection of this linkage denoting the presence of cells expressing heparin or heparan sulfate glycosaminoglycans in the biological sample.

9. Method as claimed in claim 8, characterised in that the presence of cells expressing heparin or heparan sulfate glycosaminoglycans in the biological sample indicate that the patient has a cancer or is a diagnostic of a cancer.

10. Cell penetrating polypeptide, characterised in that it is defined by the general formula (I) or (II):
X₁X₂-RK-X₃X₄-KK-X₅X₆X₇X₈X₉X₁₀-KR-X₁₁X₁₂-L-CIP (I) or
CIP-L-X₁X₂-RK-X₃X₄-KK-X₅X₆X₇X₈X₉X₁₀-KR-X₁₁X₁₂ (II),
in which X₁ to X₁₂ are defined according to claim 1, L is nothing or a spacer and CIP a cell internalization peptide.

11. Cell penetrating polypeptide according to claim 10, characterised in that it is conjugated to a biomarker at its N-terminal end or its C-terminal end or on a lateral position linked to an amino acid.

12. Cell penetrating polypeptide according to claim 10, characterised in that the spacer is a flexible linker with three atoms or a conformationally restricted linker with three atoms or an aliphatic flexible linker with five atoms or an hydrophilic flexible linker with nine atoms.

13. Cell penetrating polypeptide according to claim 12, characterised in that the spacer is an aminopentanoic acid, a polyethylene glycol, a glycine or a proline.

14. Cell penetrating polypeptide according to claim 10, characterised in that the cell internalization polypeptide CIP is defined by the sequence: SQIKIWFQNKRAKIKK (SEQ ID NO: 3), RQIKIWFQNRRMKWKK (SEQ ID NO: 4), RRWWRRWRR (SEQ ID NO: 5), RRWWRRWWR (SEQ ID NO: 6), RRWWRRWWR (SEQ ID NO: 7), RRRRRRRR (SEQ ID NO: 8), and YGRKKRRQRRR (SEQ ID NO: 9).

15. Chimeric polypeptide comprising a cell penetrating polypeptide according to claim 10 and one or more biological cargos.

16. Chimeric polypeptide according to claim 15, characterised in that the biological cargo is an acid nucleic or a polypeptide.

17. Chimeric polypeptide according to claim 15, characterised in that the biological cargo is a polypeptide defined by the sequence KRAKLAK (SEQ ID NO: 10).

18. Pharmaceutical composition, comprising a chimeric polypeptide according to claim 15 and an acceptable pharmaceutically vehicle.

19. Chimeric polypeptide according to claim 15 or a pharmaceutical composition according to claim 18 for its use as a medicament.

20. Chimeric polypeptide according to claim 15 or a pharmaceutical composition according to claim 18 for its use according to claim 19 to treat cancer involving cells expressing heparin or heparan sulfate glycosaminoglycans.


SEQUENCE LISTING (1 sequence)

SEQ ID NO: 1 - protein, 18 aa, Homo sapiens
  misc_feature (1)..(1): X is R or H or K or N or Q or W or nothing
  misc_feature (2)..(2): X is S or C or T or M or N or Q or W or nothing
  misc_feature (5)..(5): X is P or any amino acid
  misc_feature (6)..(6): X is K or H or R or W or N or Q
  misc_feature (9)..(9): X is N or D or E or Q
  misc_feature (10)..(10): X is P or any aminoacid
  misc_feature (11)..(11): X is N or D or E or Q
  misc_feature (12)..(12): X is K or H or R or N or Q or W
  misc_feature (13)..(13): X is E or D or N or Q
  misc_feature (14)..(14): X is D or N or E or Q
  misc_feature (17)..(17): X is P or any aminoacid
  misc_feature (18)..(18): X is R or K or H or N or Q or W
XXRKXXKKXXXXXXKRXX